(19) United States
(12) Patent Application Publication
(10) Pub. No.: US 2008/0070972 A1
(43) Pub. Date: Mar. 20, 2008

(54) Controlled release formulations

(76) Inventors: Irina Nikolaevna Kadiyala, Newton, MA (US); Wu Lin, Nottinghamshire (GB); Patricia Hurter, Harvard, MA (US)

Correspondence Address: Miller, Canfield, Paddock and Stone P.L.C.;c/o Jonathan P. O'Brien Ph.D., 444 West Michigan Avenue, Kalamazoo, MI, 49007, US

(21) Appl. No.: 11/807,706

(22) Filed: May 30, 2007

Related U.S. Application Data
(60) Provisional application No. 60/809,779, filed on May 31, 2006.

(51) Int. Cl. A61K 31/4025; A61P 17/06; A61P 29/00; C07D 405/12

(52) U.S. Cl. 514/422; 548/517

(57) ABSTRACT

The invention relates to a controlled release formulation for an oral cytokine inhibitor of interleukin-1 beta converting enzyme.

### CROSS-REFERENCE

This application claims the benefit of U.S. Application Ser. No. 60/809,779, filed May 31, 2006.

### BACKGROUND OF THE INVENTION

The compound of Formula (I), shown below, 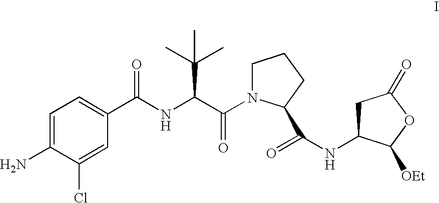
is an oral cytokine inhibitor of interleukin-1 beta converting enzyme, which is targeted at controlling the symptoms and progression of inflammatory diseases, including rheumatoid arthritis and psoriasis. The compound of Formula (I) hydrolyzes in vivo to the compound of Formula (IA), shown below.

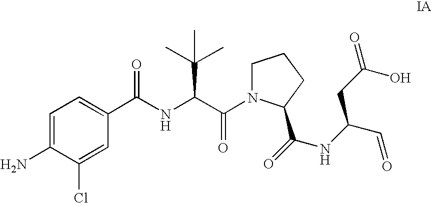

Regional absorption studies of the compound for Formula (I) indicate colonic absorption.

### SUMMARY OF THE INVENTION

In general, the invention relates to a controlled release formulation for an oral cytokine inhibitor of interleukin-1 beta converting enzyme.

In one aspect, the invention includes controlled release formulation and processes for producing the same.

In some embodiments, the process of formulating the compound of Formula (I), 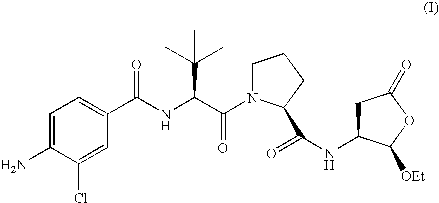
includes granulating the compound of Formula (I) in the presence of an organic solvent; wherein

(a) the stability of the compound of Formula (I) in the organic solvent is such that the solvent does not give rise to degradation of more than 5% (e.g., 2%) of the compound of Formula (I) over 24 hours at or below room temperature,

(b) the wet granulated material has an intrinsic dissolution value of less than about 0.15 mg/min/cm², and

(c) the wet granulated material has a density of between about 0.20 g/cm³ and about 0.90 g/cm³.

Embodiments of these aspects may include one or more of the following features. The density of the wet granulated material can be between about 0.50 g/cm³ and about 0.90 g/cm³. The organic solvent can be isopropyl alcohol. The compound of Formula (I) can be wet granulated with the organic solvent and a first binding agent, wherein the first binding agent can readily be dissolved in both the organic solvent used in wet granulation and in water. The first binding agent can include hydroxypropyl cellulose (e.g., between about 0.5% w/w and about 10% w/w or about 5%) or polyvinylpyrrolidone (e.g., between about 1% and about 20%). The granulation mixture can contain at least 40% (w/w) of the compound of formula I. The granulation mixture can be formulated with a second binding agent, such as polymethacrylate or ethylcellulose, a porous agent, such as lactose or mannitol, a glidant, such as talc, and a lubricant, such as sodium stearyl fumarate. The granulation mixture can be formulated with between about 0% (w/w) to about 20% (w/w) of the second binding agent; between 0% (w/w) and about 20% (w/w) of the porous agent; between about 0.1% (w/w) and about 4% (w/w) of the glidant; and between about 0.1% (w/w) and about 3% (w/w) of the lubricant. The granulation mixture can be formulated with between 5% (w/w) and about 20% (w/w) of the second binding; between about 10% (w/w) and about 20% (w/w) of the porous agent; between about 1% (w/w) and about 2% (w/w) of the glidant; and between about 0.5% (w/w) and about 3% (w/w) of the lubricant. The granulated mixture or formulated granulation mixture can be tableted for formed into pellets. The granulated mixture or formulated granulation mixture can be tableted to a hardness level between about 5 kP and about 16.0 kP.

In some aspects, the formulation can release the compound of Formula (I) for about 8 hours (e.g., +2 hours) in a dissolution test such as that described in Example 1. For example, the formulation can release between about 70% and about 90% of the compound of the Formula (I) after about 8 hours in a dissolution test. The formulation can release less than about 40%, e.g., between 5% and 40%, of the compound of Formula (I) with the first 100 minutes in an intrinsic dissolution test. The formulation can release less than about 65%, e.g., between 20% and 60%, of the compound of Formula (I) within the first 300 minutes in a dissolution test. The formulation can release between about 60% to about 80% of the compound of Formula (I) within the first 600 minutes in a dissolution test.

Specific formulations can include about 69.8% (w/w) of the compound of formula I, about 1.6% (w/w) of HPC EXF, about 10% (w/w) of Aqualon T10, about 16.5% (w/w) of Pearlitol 200 SD, about 1.4% (w/w) of Talc, and about 0.7% (w/w) of SSF. The formulated granulation mixture can include about 69.8% (w/w) of the compound of formula I, about 1.6% (w/w) of Klucel EXF, about 15% (w/w) of Eudragit RL PO, about 11.5% (w/w) of Pearlitol 200 SD, about 1.4% (w/w) of Talc, and about 0.7% (w/w) of SSF. Another formulation can include about 94.8% (w/w) of the compound of formula I, about 2.2% (w/w) of Klucel EXF, about 1.9% (w/w) of Talc, and about 1.1% (w/w) of SSF. Still another formulation can include about 95% of the compound of Formula (I) and about 5% (w/w) of HPC EXF. Yet another formulation can include about 97% of the compound of Formula (I) and about 3% (w/w) of HPC JXF.

Advantageously, the compound of Formula (I) when wet granulated, provides increased density creating improved flow properties.

As used herein, the compound of Formula (I) includes the free form of this compound and its pharmaceutically acceptable salts.

As used herein, the term “organic solvent” includes any solvent except water. Examples of an organic solvent include, without limitation, isopropyl alcohol, hexane, and acetone.

As used herein, the term “% (w/w)” refers to percentage by weight.

As used herein, the term “binding agent” (or interchangeably “retard polymer” or “matrix polymer”) refers to agents that can be used as a vehicle for delivery of the compound of Formula (I) in a controlled manner. Suitable agents may be water soluble or insoluble. Examples of the suitable agents include, without limitation, Aqualon T10 Ethylcellulose (Hercules), Klucel hydroxypropylcellulose (HPC), EXF (Hercules), Klucel HPC, JXF (Hercules), Klucel HPC, MXF (Hercules), Klucel HPC, HXF (Hercules), Eudragit RL PO, polymethacrylate (Degussa), and Avicel PH113, microcrystalcellulose (FMC).

As used herein, the term “controlled released” or “sustained release” refers to release of a drug or prodrug from its carrier at a rate or in a manner that is different from the natural rate or carrier, usually slower, and generally preferred to more linear or close to linear. When is released is extended over a long period of time (e.g., 10 hours) in a linear manner (i.e., at a rate that is closely to the same throughout the whole release process), it is also called “sustained release.”

Unless otherwise defined, all the terms herein should have the same meanings as those known in the art. All publications cited herein are incorporated in their entirety by reference.

### DETAILED DESCRIPTION OF THE DRAWINGS

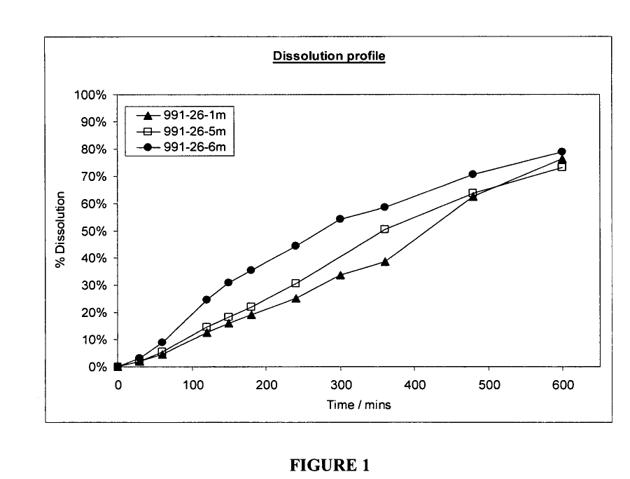

FIG. 1 illustrates the intrinsic dissolution profiles of three tablet formulations of the invention.

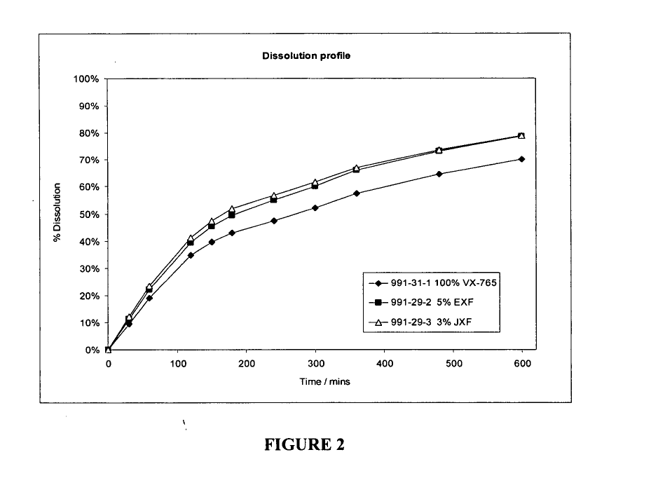

FIG. 2 illustrates the intrinsic dissolution profiles of three pellet formulations of the invention.

### DETAILED DESCRIPTION OF THE INVENTION

In general, the controlled release formulations of the present invention include compound of Formula (I) in wet granulated form. Granules of the compound of Formula (I) can be prepared by methods well known in the art. See, e.g., Pharmaceutics: the Science of Dosage Form Design, edited by Michael E. Aulton, Churchill Livingstone (Edinburgh; N.Y.), 2002; and Cyclodextrins in Pharmacy, Karl-Heinz Frömming and József Szejtli, Kluwer Academic Publishers (Dordrecht; Boston), 1994.

In some embodiments, the granules of the compound of Formula (I) are prepared by wet granulation with a non-aqueous solvent, such as an organic solvent. Generally, any organic solvent can be used for wet granulating the compound of Formula (I) provided that the resulting intrinsic dissolution value of a tablet made with the wet granulated material is less than the intrinsic dissolution value of a similar tablet formed with the neat form of the compound of formula I, e.g., the intrinsic dissolution value is less than about 0.15 mg/min/cm², e.g., about 0.095 mg/min/cm². The intrinsic dissolution value can be measured in water at 37° C. according to the U.S. Pharmacopeia (USP) guidelines, e.g., as provided at http://www.usp.org/.

In some embodiments, at least about 50% of the compound of Formula (I) is released from the granules described above within the first 10 hours in an in vitro dissolution test as measured by a method known in the art. An example of applicable method is a USP protocol for a delayed release formulations in Apparatus 2 (see, e.g., http://www.usp.org.). An alternative method includes using the paddle method at 50 rpm for up to 10 hours. In still another method, the in vitro dissolution values can be determined by using HPLC to assess the dissolved amounts of the compound of Formula (I) and metabolites thereof. The amount of the released compounds can be determined, e.g., by comparing the total peaks area with that of an external control. Examples of columns that can be used in this method include a Waters Symmetry C-18, 50×4.6 mm, 3.5 μm column (PN# WAT200625) with mobile phase composed of water, methanol, acetonitrile, and pH 5.0 phosphate buffer, under gradient elution conditions. Detection can be performed by ultraviolet (UV) absorbance at 280 nm. The resulting samples from either method can be analyzed by HPLC. Other methods known in the art can also be used to obtain the release profile of the granules of the compound of Formula (I) or each formulation containing the compound of formula I. See, e.g., Controlled Drug Delivery Fundamentals and Applications, 2^{nd} Edition, J. R. Robinson and V. H. L. Lee, Marcel Dekker (New York), 1987. For instance, a sample of the formulation can be placed in an aqueous medium and the concentration of the compound of Formula (I) in the medium can be determined, e.g., by measured the activity of interleukin-1 beta converting enzyme, a target enzyme of the compound of formula I.

In other embodiments, any organic solvent can be used to wet granulate the compound of Formula (I) provided that (a) the stability of the compound of Formula (I) in the organic solvent is such that the solvent does not give rise to degradation of more than 5% of the compound of Formula (I) over 24 hours at or below room temperature, (b) the wet granulated material has an intrinsic dissolution value of less than about 0.15 mg/min/cm², and (c) the wet granulated material has a density of between about 0.20 g/cm³ and about 0.90 g/cm³, e.g., between about 0.5 and about 0.65 g/cm³. The increase in density of the granulated material relative to the non-granulated material results in improved flow characteristics which facilitate easier handling during manufacturing processes such as formulation. Bulk and granulated densities can be measured with a measuring cylinder. The values for the bulk (ρb) and tap (ρt) densities can be used to calculate index of compressibility (% Compressibility), which is indicative of the flow properties of the material. The % Compressibility can be calculated by the following formula: % Compressibility=[(ρt−−ρb)/ρt-]×100%. See, e.g., The theory and practice of industrial pharmacy, by L. Lachman, H. Lieberman, and J. Kanig, in Varghese Publishing House, 3rd Indian Edition, (Hind Rajasthan Bldng, Dadar Bombay 400 014), 1987, p. 184. Also, flowability of the material can be determined by Flodex analysis. The Flodex Test Apparatus is ideal for determining the intrinsic flowability of powders (which is recommended for quality control of powders). The Flodex instrument includes a complete set of index flow disks with opening of different diameter. A tested powder is allowed to flow through the opening. The ability of the powder to flow through the opening is indicative of the flow properties of the tested powder. See, e.g., Composite Method to Quantify Powder Flow as a Screening Method in Early Tablet or Capsule Formulation Development, by Michael K. Taylor, Jeri Ginsburg, Anthony Hickey, and Ferdous Gheyas, in AAPS PharmSciTech, 2000, 1(3): article 18.

In certain embodiments, the compound of Formula (I) is wet granulated with isopropyl alcohol (IPA).

In conventional wet granulation approaches, most tablet excipients such as fillers, binders and release retard polymers are normally blended with the drug material before granulation. Unlike conventional methods, the material of the compound of Formula (I) is wet granulated prior to full blending with all of the formulation excipients to overcome the low density and poor flowability of the non-granulated material by producing granules with significantly improved density and flowability. The resulting granules are easier to blend with different matrix polymers and other excipients to produce controlled release formulations.

In some embodiments, the compound of Formula (I) is wet granulated with an organic solvent, such as IPA, and the resulting granules are blending with the excipients and other components of the controlled release formulation. In other embodiments, the compound of Formula (I) and first binding agent are wet granulated. Yet in some other embodiments, the compound of Formula (I) can be granulated with the solvent without any binding agents.

The first binding agent can be any agent that dissolves in both the organic solvent used in wet granulation and in water. Examples of the first binding agent include, without limitation, hydroxypropyl cellulose (HPC) and polyvinylpyrrolidone (PVP).

The amount of first binding agent used in the wet granulation can be any amount which provides resulting granules having a size ranging between about 150 μm to about 500 μm, containing greater than about 90% of the compound of formula I, and a density between 0.35 and about 0.70 g/cm³. In certain embodiments, the compound of Formula (I) is wet granulated with Klucel HPC EXF or PVP (10% in a solution of IPA). In some embodiments, the compound of Formula (I) is wet granulated with between about 0.5% w/w to about 10% w/w, e.g., about 5% w/w, of Klucel HPC EXF or with between about 1% and about 20%, e.g., about 10%, PVP in the solvent, IPA.

Before granulation, the bulk material of the compound of Formula (I) can be sieved, e.g., by using a 500 μm sieve.

In some embodiments, granules of the compound of Formula (I) can be mixed with a binding agent and, optionally, other known pharmaceutical excipients (e.g., other binding agents, a porous agent, and a filler) to obtain a pharmaceutical formulation. When administered in an aqueous medium, such a pharmaceutical formulation is generally capable of releasing the compound of Formula (I) in a controlled manner. Further, depending on the type of the binding agents and other excipients and the weight ratio of the granules of the compound of Formula (I) and the binding agents and other excipients, the pharmaceutical formulation can provide a sustained release of the compound of Formula (I) at a certain level over an extended period of time, e.g., for at least 8 hours or between 8 and 10 hours, such that between about 70% and about 90% of the compound of the Formula (I) is released. The precise release profile of the compound of Formula (I) can be changed by adjusting the type and amount of the binding agents and other excipients contained in the formulation, the hardness of the tablets and pellets. In some embodiments, the controlled release formulations provide release of less than about 40%, e.g., between about 5% to about 40%, of the compound of Formula (I) in the first 100 minutes after placement in a dissolution medium as measured via the dissolution methodology described herein. In certain aspects of these embodiments, the controlled release formulations provide release of less than about 40%, e.g., between about 5% to about 40%, of the compound of Formula (I) in the first 100 minutes; and release of less than about 65%, e.g., between about 20% to about 60%, of the compound of Formula (I) in the first 300 minutes. In still other aspects of these embodiments, the controlled release formulations provide release of less than about 40%, e.g., between about 5% to about 40%, of the compound of Formula (I) in the first 100 minutes; release of less than about 65%, e.g., between about 20% to about 60%, of the compound of Formula (I) in the first 300 minutes; and release of between about 60% to about 80%, of the compound of Formula (I) in the first 600 minutes. In other embodiments, the controlled release formulations provide release of less than about 25%, e.g., between about 5% to about 20%, of the compound of Formula (I) in the first 100 minutes.

Still in some embodiments, the process of formulating granules containing the compound of Formula (I) may be conducted without the presence of an organic or aqueous solvent, i.e., by dry granulation. As a result, the granules thus obtained may contain about 100% (w/w) of the compound of Formula (I), which may also posses the same features or properties as discussed herein

The pharmaceutical formulation can be processed into the form of tablets or pellets for oral administration. The tablets and pellet formulations contain between about 300 mg to about 1000 mg of the compound of formula I.

The sustained release formulations which provide near linear release of the compound of Formula (I) over about 10 hours includes pellets or tables of granules resultant from wet granulation of the compound of Formula (I) with less than about 3% w/w hydroxypropyl cellulose. In some embodiments, the tablets containing the sustained release formulations have a hardness less than about 10 kP, e.g., the tables have a hardness between about 6.5 and about 9.0 kP. In certain embodiments, the tablet hardness is between about 7.0 kP and about 9.0 kP.

The formulation may also include additional excipients including, without limitations, additional binding agents, e.g., polymethacrylate or ethylcellulose; porous agents, e.g., lactose or mannitol; glidants, e.g., talc; and lubricants, e.g., sodium stearyl fumarate (SSF).

In some embodiments, the formulation containing wet granulated compound of Formula (I) includes a second binding agent in an amount between about 0% (w/w) to about 20% (w/w), e.g., between 5% (w/w) to about 20% (w/w); a porous agent in an amount between 0% (w/w) to about 20% (w/w), e.g., between about 10% (w/w) to about 20% (w/w); a glidant in an amount between about 0.1% (w/w) to about 4% (w/w), e.g., between about 1% (w/w) to about 2% (w/w); and a lubricant in an amount between about 0.1% (w/w) to about 3% (w/w), e.g., between about 0.5% (w/w) to about 1.5% (w/w).

Not intended to limit the scope of the claimed invention, examples of the invention are set forth below for illustration purpose.

### EXAMPLE 1

### Dissolution Values

The dissolution test method was derived from the USP dissolution monograph for modified release dosage forms. Apparatus 2 (paddle method) was employed at 50 rpm for up to 10 hours. Analysis of the resultant samples was performed by HPLC.

The HPLC method assessed the amount of the compound of Formula (I) and metabolites thereof present in dissolution samples, using a Waters Symmetry C-18, 50×4.6 mm, 3.5 □m column (Part No. WAT200625) with mobile phase composed of water, methanol, acetonitrile, and phosphate buffer pH 5.0, under gradient elution conditions. At a flow of 2.5 mL/minute and a column temperature of 45° C., the retention time of the compound of Formula (I) and metabolites thereof was approximately 3.7 minutes and 1.5 minutes respectively. Detection was performed by ultraviolet (UV) absorbance at 280 nm. The release of the compound of Formula (I) was calculated by comparison of the total peak area of the compound of Formula (I) and metabolites thereof and with an external standard solution of the compound of formula I.

### EXAMPLE 2

### Bulk Non-Granulated Materials

The bulk material of the compound of Formula (I) was found as white powder with some aggregates. The material was first sieved through a 500 μm sieve. The density of the 500 μm sieved bulk material was found to be around 0.24 g/cm³ with very poor flowability. The morphology of the material was investigated under an optical microscope and it was found that majority of the bulk material are aggregates from much smaller crystals. These aggregates are highly porous and hence generate a low apparent density and poor flowability, which makes the direct compression for tableting very difficult.

### EXAMPLE 3

### Wet Granulation of the Compound of Formula (I)

To prepare the Compound of Formula (I) granules by wet granulation, bulk material of the compound of Formula (I) (500 μm sieved) can be placed in a mortar and a first binding agent (e.g., HPC EXF at 5% w/w or PVP at 10% w/w) solution in isopropyl alcohol (IPA) is then added dropwise and mixed using a pestle until the end point of granulation is reached. The mass is then manually passed through a sieve (e.g., 1.18 mm) and dried at room temperature overnight. The granules are then passed through a finer sieve (e.g., 500 μm) to break any aggregates. The large particles can be milled by using a mortar and pestle, and again sieved through the finer sieve. The granules thus prepared are then sieved again to remove the fraction of powder that is, e.g., smaller than 150 μm. Bulk and granule densities and flowability of the final granules can be examined as described above. The granules of the Compound of Formula (I) are stored in an amber bottle at room temperature.

Compared to the bulk form of the Compound of formula I, granules produced from wet granulation have higher density and better flowability and thus are more suitable for high-dosage administration. For instance, depending on the amount of the binding agent used in granulation and the length of the granulation, the density of granules of the Compound of Formula (I) can be increased to, e.g., at least 0.4 g/cm³ (e.g., 0.5-0.65 g/cm³), from about 0.2 g/cm³ of the natural form.

### EXAMPLE 4

### Preparation of Tablets for Controlled Release of the Compound of Formula (I)

The following tablets were prepared using the wet granulated material and the excipients:

| Tablet | Composition/tablet | Tablet size | Hardness |
|---|---|---|---|
| 991-26-1 | Wet Granulation: the compound | 18 × 9 mm | 7.6 KP |
| | of Formula (I) (600 mg, 69.8%), | 860 mg | |
| | and HPC EXF (1.6%) with IPA, | | |
| | Aqualon T10 (10.0%), | | |
| | Pearlitol 200 SD (16.5%), | | |
| | Talc (1.4%), | | |
| | SSF (0.7%) | | |
| 991-26-5 | Wet Granulation: the compound | 18 × 9 mm | 8.6 KP |
| | of Formula (I) (600 mg, 69.8%) | 860 mg | |
| | and Klucel EXF (1.6%) with IPA, | | |
| | Eudragit RL PO (15.0%), | | |
| | Pearlitol 200 SD (11.5%), | | |
| | Talc (1.4%), | | |
| | SSF (0.7%) | | |
| 991-26-6 | Wet Granulation: the compound | 18 × 9 mm | 7.8 KP |
| | of Formula (I) (600 mg, 94.8%) | 633 mg | |
| | and Klucel EXF (2.2%) with IPA, | | |
| | Talc (1.9%), | | |
| | SSF (1.1%) | | |

FIG. 1 illustrates the dissolution profiles for 10 hours. The three tablets exhibited good dissolution profiles, i.e., nearly linear, releasing between about 70% to about 80% of the compound of Formula (I) over the 10-hour period.

### EXAMPLE 5

### Preparation of Pellet Formulations

Pellets were prepared at a 30-gram scale. The size fraction of the pellet formulation was 800/1180 μm. The details of the pellet formulations are included in the following table. The density of the final pellets was examined and the pellets were stored in a vial before the dissolution test.

The following pellets were prepared using the wet granulated material as indicated below:

| | | Density of |
|---|---|---|
| Formulation | Ingredients | pellets (g/cm³) |
| 991-29-2 | Wet granulation of the | 0.51 |
| | compound of Formula (I) with | |
| | HPC EXF (95:5) in IPA | |
| 991-29-3 | Wet granulation of the | 0.62 |
| | compound of Formula (I) with | |
| | HPC JXF (97:3) in IPA | |
| 991-31-1 | Wet granulation of the | 0.61 |
| | compound of Formula (I) with | |
| | IPA | |

FIG. 2 illustrates the dissolution profiles of Formulations 991-29-2, 991-29-3, and 991-31-1 for 10 hours. The three pellet formulations exhibited good dissolution profiles, i.e., quasi-linear, releasing between about 60% to about 80% of the compound of Formula (I) over the 10 hour period.

### OTHER EMBODIMENTS

It is to be understood that while the invention has been described in conjunction with the detailed description thereof, the foregoing description is intended to illustrate and not limit the scope of the invention, which is defined by the scope of the following claims. Other aspects, advantages, and modifications are within the scope of the present invention

What is claimed is:

1. A process of formulating the compound of Formula (I), 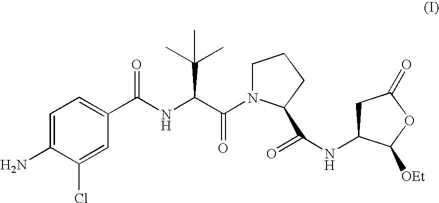
comprising:
granulating the compound of Formula (I) in the presence of an organic solvent; wherein
(a) the stability of the compound of Formula (I) in the organic solvent is such that the solvent does not give rise to degradation of more than 5% of the compound of Formula (I) over 24 hours at or below room temperature,
(b) the wet granulated material has an intrinsic dissolution value of less than about 0.15 mg/min/cm², and
(c) the wet granulated material has a density of between about 0.20 g/cm³ and about 0.90 g/cm³.

2. The process of claim 1, wherein the density of the wet granulated material is between about 0.50 g/cm³ to about 0.90 g/cm³.

3. The process of claim 1, wherein the organic solvent is isopropyl alcohol.

4. The process of claim 1, wherein the compound of Formula (I) is wet granulated with the organic solvent and a first binding agent, wherein the first binding agent dissolves both in the organic solvent used in wet granulation and in water.

5. The process of claim 4, wherein the first binding agent comprises hydroxypropyl cellulose or polyvinylpyrrolidone.

6. The process of claim 5, wherein the wet granulated material comprises between about 0.5% w/w and about 10% w/w hydroxypropyl cellulose.

7. The process of claim 6, wherein the wet granulated material comprises about 5% w/w hydroxypropyl cellulose.

8. The process of claim 5, wherein the wet granulated material comprises between about 1% and about 20% polyvinylpyrrolidone in the organic solvent.

9. The process of claim 1, wherein the formulated granulation mixture contains at least 40% (w/w) of the compound of Formula (I) by weight

10. The process of claim 9, wherein the formulated granulation mixture contains at least 85% (w/w) of the compound of Formula (I) by weight.

11. The process of claim 10, wherein the formulated granulation mixture comprises 100% of the compound of formula I.

12. The process of claim 1, further comprising formulating the granulation mixture with a second binding agent.

13. The process of claim 12, wherein the second binding agent comprises polymethacrylate or ethylcellulose.

14. The process of claim 1, further comprising formulating the granulation mixture with a porous agent.

15. The process of claim 14, wherein the porous agent comprises lactose or mannitol.

16. The process of claim 1, further comprising formulating the granulation mixture with a glidant.

17. The process of claim 16, wherein the glidant comprises talc.

18. The process of claim 1, further comprising formulating the granulation mixture with a lubricant.

19. The process of claim 18, wherein the lubricant comprises sodium stearyl fumarate.

20. The process of claim 1, further comprising formulating the granulation mixture with a second binding agent, a porous agent, a glidant, and a lubricant.

21. The process of claim 20, wherein the second binding agent comprises polymethacrylate or ethylcellulose, the porous agent comprises lactose or mannitol, the glidant comprises talc, and the lubricant comprises sodium stearyl fumarate.

22. The process of claim 21, wherein the granulation mixture is formulated with between about 0% (w/w) to about 20% (w/w) of the second binding agent; between 0% (w/w) to about 20% (w/w) of the porous agent; between about 0.1% (w/w) to about 4% (w/w) of the glidant; and between about 0.1% (w/w) to about 3% (w/w) of the lubricant.

23. The process of claim 22, wherein the granulation mixture is formulated with between 5% (w/w) to about 20% (w/w) of the second binding; between about 10% (w/w) to about 20% (w/w) of the porous agent; between about 1% (w/w) to about 2% (w/w) of the glidant; and between about 0.5% (w/w) to about 1.5% (w/w) of the lubricant.

24. The process of claim 1, wherein the formulated granulation mixture comprises about 69.8% of the compound of Formula (I), about 1.6% of HPC EXF, about 10% Aqualon T10, about 16.5% Pearlitol 200 SD, about 1.4% Talc, and about 0.7% SSF.

25. The process of claim 1, wherein the formulated granulation mixture comprises about 69.8% of the compound of formula I, about 1.6% Klucel EXF, about 15% Eudragit RL PO, about 11.5% Pearlitol 200 SD, about 1.4% Talc, and about 0.7% SSF.

26. The process of claim 1, wherein the formulated granulation mixture comprises about 94.8% of the compound of formula I, about 2.2% Klucel EXF, about 1.9% Talc, and about 1.1% SSF.

27. The process of claim 1, wherein the formulated granulation mixture comprises about 95% of the compound of Formula (I) and about 5% of HPC EXF.

28. The process of claim 1, wherein the formulated granulation mixture comprises about 97% of the compound of Formula (I) and about 3% of HPC JXF.

29. The process of claim 1, wherein the granulated mixture or formulated granulation mixture is tableted to a hardness between about 6.5 kP to about 16.0 kP.

30. The process of claim 1, further comprising forming pellets with the granulated mixture or formulated granulation mixture.

31. The process of claim 1, wherein the formulation releases the compound of Formula (I) after about 8 hours in a dissolution test.

32. The process of claim 1, wherein the formulation releases between about 70% and about 90% of the compound of the Formula (I) after about 8 hours in a dissolution test.

33. The process of claim 1, wherein the formulation releases less than about 40% of the compound of Formula (I) within the first 100 minutes in a dissolution test.

34. The process of claim 33, wherein the formulation releases between about 5% and about 40% of the compound of Formula (I) within the first 100 minutes in a dissolution test.

35. The process of claim 1, wherein the formulation releases less than about 65% of the compound of Formula (I) within the first 300 minutes in a dissolution test.

36. The process of claim 35, wherein the formulation releases between about 20% to about 60% of the compound of Formula (I) within the first 300 minutes in a dissolution test.

37. The process of claim 1, wherein the formulation releases between about 60% to about 80% of the compound of Formula (I) within the first 600 minutes in a dissolution test.

38. The process of claim 1, wherein at least 50% of the compound of Formula (I) is released from the granulated mixture or formulated granulation mixture after the first 10 hours in a dissolution test.

39. A controlled release formulation, comprising a wet granulated material which comprises the compound of Formula (I), 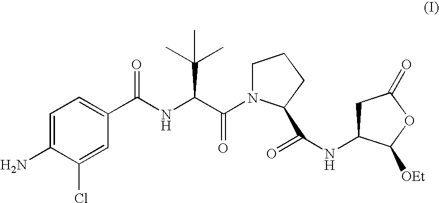
wherein the wet granulated material exhibits an intrinsic dissolution value of less than about 0.15 mg/min/cm², and a density between about 0.20 g/cm³ and about 0.90 g/cm³.

40. The controlled release formulation of claim 39, wherein the wet granulated material exhibits a density between about 0.50 g/cm³ and about 0.90 g/cm³.

41. The controlled release formulation of claim 39, wherein the wet granulated material further comprises a first binding agent, wherein the first binding agent dissolves in both the organic solvent used in wet granulation and in water.

42. The controlled release formulation of claim 39, wherein the first binding agent comprises hydroxypropyl cellulose or polyvinylpyrrolidone.

43. The controlled release formulation of claim 42, wherein the wet granulated material comprises between about 0.5% (w/w) and about 10% (w/w) of hydroxypropyl cellulose.

44. The controlled release formulation of claim 43, wherein the wet granulated material comprises about 5% (w/w) of hydroxypropyl cellulose.

45. The controlled release formulation of claim 42, wherein the wet granulated material comprises between about 1% (w/w) and about 20% (w/w) polyvinylpyrrolidone.

46. The controlled release formulation of claim 39, wherein the wet granulated material contains at least 85% (w/w) of the compound of formula I.

47. The controlled release formulation of claim 39, further comprising a second binding agent.

48. The controlled release formulation of claim 47, wherein the second binding agent comprises polymethacrylate or ethylcellulose.

49. The controlled release formulation of claim 47, further comprising a porous agent.

50. The controlled release formulation of claim 49, wherein the porous agent comprises lactose or mannitol.

51. The controlled release formulation of claim 47, further comprising a glidant.

52. The controlled release formulation of claim 51, wherein the glidant comprises talc.

53. The controlled release formulation of claim 47, further comprising a lubricant.

54. The controlled release formulation of claim 53, wherein the lubricant comprises sodium stearyl fumarate.

55. The controlled release formulation of claim 39, further comprising a second binding agent, a porous agent, a glidant, and a lubricant.

56. The controlled release formulation of claim 55, wherein the formulation comprises between about 0% (w/w) and about 20% (w/w) of the second binding agent; between 0% (w/w) and about 20% (w/w) of the porous agent; between about 0.1% (w/w) and about 4% (w/w) of the glidant; and between about 0.1% (w/w) and about 3% (w/w) of the lubricant.

57. The controlled release formulation of claim 56, wherein the formulation comprises between 5% (w/w) and about 20% (w/w) of the second binding; between about 10% (w/w) and about 20% (w/w) of the porous agent; between about 1% (w/w) and about 2% (w/w) of the glidant; and between about 0.5% (w/w) and about 1.5% (w/w) of the lubricant.

58. A controlled release formulation, comprising about 69.8% (w/w) of the compound of Formula (I), about 1.6% (w/w) of HPC EXF, about 10% (w/w) of Aqualon T10, about 16.5% (w/w) of Pearlitol 200 SD, about 1.4% (w/w) of Talc, and about 0.7% (w/w) of SSF, wherein the compound of Formula (I) and HPC EXF are wet granulated with isopropyl alcohol.

59. A controlled release formulation, comprising about 69.8% (w/w) of the compound of Formula (I), about 1.6% (w/w) of Klucel EXF, about 15% (w/w) of Eudragit RL PO, about 11.5% (w/w) of Pearlitol 200 SD, about 1.4% (w/w) of Talc, and about 0.7% (w/w) of SSF, wherein the compound of Formula (I) and Klucel EXF are wet granulated with isopropyl alcohol.

60. A controlled release formulation, comprising about 94.8% (w/w) of the compound of Formula (I), about 2.2% (w/w) of Klucel EXF, about 1.9% (w/w) of Talc, and about 1.1% (w/w) of SSF, wherein the compound of Formula (I) and Klucel EXF are wet granulated with isopropyl alcohol

61. The controlled release formulation of claim 39, wherein the mixture is tableted to a hardness of less than about 16 kP.

62. The controlled release formulation of claim 61, wherein the mixture is tableted to a hardness of between 6.5 kP and about 9.0 kP.

63. A controlled release formulation, comprising pellets including wet granulated material, wherein the wet granulated material comprises 95% of the compound of Formula (I) and about 5% of HPC EXF granulated in isopropyl alcohol.

64. A controlled release formulation, comprising pellets including wet granulated material, wherein the wet granulated material comprises about 97% of the compound of Formula (I) and about 3% of HPC JXF granulated in isopropyl alcohol.

65. The controlled release formulation of claim 39, wherein the formulation releases between about 70% and about 90% of the compound of the Formula (I) after about 8 hours in dissolution test.

66. The controlled release formulation of claim 39, wherein the formulation releases less than about 40% of the compound of Formula (I) within the first 100 minutes in a dissolution test.

67. The controlled release formulation of claim 66, wherein the formulation releases between about 5% and about 40% of the compound of Formula (I) within the first 100 minutes in a dissolution test.

68. The controlled release formulation of claim 39, wherein the formulation releases less than about 65% of the compound of Formula (I) within the first 300 minutes in a dissolution test.

69. The controlled release formulation of claim 68, wherein the formulation releases between about 20% and about 60% of the compound of Formula (I) within the first 300 minutes in a dissolution test.

70. The controlled release formulation of claim 39, wherein at least 50% of the compound of Formula (I) is released from the formulation within the first 10 hours in a dissolution test.

71. The controlled release formulation of claim 70, wherein the formulation releases between about 60% and about 80% of the compound of Formula (I) within the first 600 minutes in a dissolution test.
